(19) United States
(12) Patent Application Publication
(10) Pub. No.: US 2004/0267112 A1
(43) Pub. Date: Dec. 30, 2004

(54) Methods for association of markers and uses of the methods

(76) Inventors: Karl Barth, Hochstadt (DE); Gerd Wessels, Effeltrich (DE)

Correspondence Address: SCHIFF HARDIN, LLP, PATENT DEPARTMENT, 6600 SEARS TOWER, CHICAGO, IL, 60606-6473, US

(21) Appl. No.: 10/865,087

(22) Filed: Jun. 10, 2004

(30) Foreign Application Priority Data
Jun. 11, 2003 (DE) 103 26 281.4

(51) Int. Cl. A61B 5/05

(52) U.S. Cl. 600/424

(57) ABSTRACT

A method associates N markers arranged on a subject and K markers mapped in an image of the subject relative to one another. In a coordinate system associated with the subject, the coordinates of the N markers arranged on the subject are determined, and subsequently the distance sums of each of the N markers are determined relative to the remaining N markers. The distance sums are also sorted in ascending/descending order. A first sequence of the N markers arranged on the subject is subsequently determined based on the ascending or, respectively, descending order of the distance sums. A second sequence of the K markers mapped in the image is determined analogously. Finally, the first sequence of the N markers is associated with the second sequence of the K markers. Alternatively, an association of markers mapped in two images can ensue in a corresponding manner.

### BACKGROUND OF THE INVENTION

The invention concerns methods for association of N markers arranged on a subject and K markers mapped in an image of the subject relative to another. The invention moreover concerns methods for an association of N markers mapped in a first image and K markers mapped in a second image relative to one another. The invention also concerns uses of these methods.

In a clinical workflow, image data sets (acquired, for example, with an x-ray apparatus) or, for example, volume data sets (acquired with a computer tomograph or a magnetic resonance apparatus) are often acquired at different locations and time-delayed. This creates the problem of producing a precise spatial reference between two various data sets that registers the typically ensuing spatial displacements and rotations. The determination of the precise spatial reference is implemented with what is known as the registration. For example, via registered coordinates, it is possible to directly superimpose various mappings or, for example, to mix the position of a stereotactical instrument into a three-dimensional image acquired beforehand. Another application is to respectively, precisely provide the corresponding computer tomography view at a current endoscopic, laparoscopic or ultrasound image, or even to superimpose both views.

In the registration of markers, the markers are applied to the subject, e.g., a patient. Should a procedure ensue on the patient, the markers are preferably applied in the surroundings of the operation area. Positions are preferably selected that, for example, optimally move little as a consequence of the respiration of the patient. The markers are therefore preferably arranged in the vicinity of skull or pelvic bones.

Should, for example, the medical instrument with which the procedure is implemented on the patient be mixed into an image of the patient created before the procedure, the markers are arranged on the patient before the exposure and remain on the patient until the end of the procedure. Alternatively, the positions at which the markers are arranged for the exposure can also be identified with a painted crosshair or a color dot in order to later reattach possibly removed markers at their locations, or the painted marks themselves serve as markers during the procedure.

For the actual registration, each marker arranged on the patient must be currently identified in its position, for example via manual preparation in a magnetic field or, for example, via optical recognition of its position in space. The same is true for the registration for the markers mapped in the image, whereby here in turn an automatic detection is possible. Via the identification in both systems, the reference of the coordinates of the coordinate system of the image with that of the coordinate system of the navigation system in whose field the patient lies during the procedure is produced. A transformation matrix is calculated from this association, by which the registration is completed.

Should two images of a subject that have been acquired with two different imaging apparatuses be superimposed or fused, a reference is likewise necessary between both coordinate systems of both images. This reference is likewise produced in that the mapped markers in both images are manually prepared or automatically detected. The transformation matrix is calculated based on the association of the markers mapped in both of the images.

According to a first known method, using the coordinates of the quantity O of markers arranged on the subject with regard to the first coordinate system or, respectively, the quantity Q of markers mapped in the image of the subject with regard to the second coordinate system, information about the sequence of the markers arranged on the subject and the mapped markers corresponding with one another can be found in that, for example, a specific series of the coordinates 1 through N from the quantity O is isolated/singled out and all permutations are formed from the markers 1 through K from the quantity Q. The difference of each point distance in the quantity O from the corresponding point distance in the respective permutation is formed from the quantity Q. The matching association of the markers 1 . . . K is those permutations of the quantity Q for which the sum of the contributions of the distance differences from the distances from the quantity O is smallest. The transformation matrix can subsequently be calculated using the coordinates of the markers or, respectively, the mapped markers in the respective coordinate systems for the quantity O and the quantity Q.

A further method is specified in German Patent Document No. DE 199 28 737 C1 which saves the elaborate series of the markers in a fixed sequence. An unambiguous sequence of the markers can be acquired in all representations via direct point distances by way of a recursive method.

It has proven to be disadvantageous that here as well, for example, given a larger number of markers, the number of the permutations significantly grows factorially. This leads to an increase of the calculation time for the determination of the sequences of markers corresponding with one another.

### SUMMARY OF THE INVENTION

The invention is based on the object to execute methods for association of N markers arranged on a subject and K markers mapped in an image of the subject relative to another, such that the reliability of the registration is increased.

It is a further object of the invention to execute methods for association of N markers mapped in a first image and K markers mapped in a second image relative to one another, such that the reliability of the registration is increased.

Further objects of the invention are the specifications of uses of these methods.

The first object of the invention is achieved via methods for association of set O of N markers arranged on a subject and set Q of K markers mapped in an image of the subject relative to one another, comprising the following method steps:

determination of the coordinates of the N markers arranged on the subject in a first coordinate system,

determination of a first set of distance sums in which

respectively, the distance sum of each marker arranged on the subject is added to each of the remaining N−1 markers arranged on the subject, and

these distance sums are sorted in ascending order,

determination of a first sequence of the N markers arranged on the subject based on the ascending order of the distance sums of the first set of distance sums.

determination of the coordinates of the K markers mapped in the image in a second coordinate system,

determination of a second set of distance sums in which

respectively, the distance sum of each marker mapped in the image is added to each of the remaining K−1 markers mapped in the image, and

these distance sums are sorted in ascending order,

determination of a second sequence of the K markers mapped in the image based on the ascending order of the distance sums of the second set of distance sums, and

in the event that N=K, then association of the first sequence of the N markers with the second sequence of the K mapped markers.

Both sets of distance sums can alternatively also be formed such that the corresponding distance sums are sorted in descending order.

The sum of the distances to the remaining markers arranged on the subject is initially inventively determined for each marker of the markers arranged on the subject. These distance sums are subsequently sorted in ascending or, respectively, descending order. If the distance sums are, for example, sorted in ascending order, the first distance sum of the first set of distance sums is thus the smallest distance sum. The subsequent distance sums of the first set then always increase, such that the largest distance sum is the last distance sum of the set.

The sequence of the distance sums of the first set O is henceforth also the sequence of the treatment of the N markers arranged on the subject, whereby for the ascending sequence, one starts with the marker that is associated with the smallest distance sum.

For each marker mapped in the image, the sum of the distances to the remaining markings mapped in the image is subsequently determined. These distance sums are afterwards sorted in ascending or descending order.

The sequence of the distance sums of the second set Q is henceforth also the sequence of the treatment of the K markers mapped in the image, whereby for the ascending sequence, one starts with the marker that is associated with the smallest distance sum.

If precisely as many markers are arranged on the subject as are mapped in the image, i.e., if N=K, then the association of the markers arranged on the subject with the corresponding markers mapped in the image results based on the sequence of the sorted markers or the sorted mapped markers.

The second object of the invention is achieved via a method for association of set O of N markers mapped in a first image and set Q of K markers mapped in a second image relative to one another, whereby both images are mappings of a subject and the markers are arranged on the subject during the exposures and are mapped in both images, comprising the following method steps:

determination of the coordinates of the N markers mapped in the first image in a first coordinate system,

determination of a first set of distance sums in which

respectively, the distance sum of each marker mapped in the first image is added to each of the remaining N−1 markers mapped in the first image, and

these distance sums are sorted in ascending order,

determination of a first sequence of the N markers mapped in the first image based on the ascending order of the distance sums of the first set of distance sums

determination of the coordinates of the K markers mapped in the second image in a second coordinate system,

determination of a second set of distance sums in which

respectively, the distance sum of each marker mapped in the second image is added to each of the remaining K−1 markers mapped in the second image, and

these distance sums are sorted in ascending order,

determination of a second sequence of the K markers mapped in the second image based on the ascending order of the distance sums of the second set of distance sums, and

in the event that N=K, then association of the first sequence of the N mapped markers with the second sequence of the K mapped markers.

Both sets of distance sums can alternatively also be formed such that the corresponding distance sums are sorted in descending order.

Instead of an association of markers arranged on the subject with the markers mapped in the image, this method generates an association of markers mapped in two images.

According to an embodiment of the invention, for the improbable, but possible, case of equal distance sums, possible duplicate (spatially equal) points are removed and the remaining points are sorted such that a straight line is formed from the first unambiguous point to the points with identical distance sums, and the sorting is implemented such that the length of the straight line changes monotonically. Should identical lengths result, they are sorted such that the solid angle of the straight lines increases or decreases monotonically in a defined mathematical system. “Monotonically” means, for example, increasing line lengths or, respectively, positively increasing angles. “Defined mathematical system” means, for example, a Cartesian rule system.

If the number N of the points of the set O is unequal to the number K of the points of the set Q because, for example, fewer markers are mapped than are arranged on the subject/in the other image, or because one marker or multiple markers that are mapped in the image have fallen off the subject, it must additionally be determined which marker or markers in the larger of the two sets is (are) not contained in the other set. For example, according to an embodiment of the inventive method, for N<K, the following method steps are therefore implemented:

formation of partial sequences from the second sequence, whereby the number of mapped markers of each partial sequence is equal to N,

determination of the distances between the coordinates of two adjacently mapped markers of the partial sequences,

determination of the correlation coefficients for each of the partial sequences with the first sequence, in that respectively the distances between two mapped markers of a partial sequence are correlated with the corresponding distances between two markers or between two mapped markers of the first sequence, and

association of the first sequence with that partial sequence with the largest correlation coefficients.

According to a further embodiment, for example, for N<K, the following method steps can also be implemented:

formation of partial sequences from the second sequence, whereby the number of mapped markers of each partial sequence is equal to N,

determination of the distances between the coordinates for each two adjacently mapped markers of the partial sequences,

determination of the correlation coefficients for each of the partial sequences with the first sequence, in that respectively the distance sums between two mapped markers of a partial sequence are correlated with the corresponding distance sums between two markers or between two mapped markers of the first sequence, and

association of the first sequence with that partial sequence with the largest correlation coefficients.

The partial sequences are thus acquired from that sequence of both sequences that has the larger number of points, thus markers or mapped markers. The partial sequence with the largest correlation coefficients (which, given more precise coincidence, is 1.0) then comprises only those points which also correspond to points in the smaller set. Moreover, a suitable correspondence also equally results.

For a further processing, it can be appropriate to determine a scale ratio between both coordinate systems according to the determination of the equally large point sets. The scale ratio can, for example, be calculated from the ratio of the largest distance of two markers arranged on the subject to the largest distance of two markers mapped in the image, or from the ratio of the distance sums of the subject markers to the distance sums of the image markers.

The scale ratio can, for example, be used for plausibility monitoring. Thus, an embodiment of the inventive method implements the following method steps when N=K, under consideration of the scale factor:

comparison of corresponding distance sums of both sets of distance sums, and

generation of an error message when at least one pair of corresponding distance sums differs by more than a predetermined first measure.

An improvement also results when, according to an embodiment of the invention (for example for N<K), the following method steps are implemented:

starting from the first sequence and taking into account the scale factor, successive comparison of the distances of two successive markers of the first sequence with the distances of subsequent markers of the second sequence, and

removal of the current subsequent markers from the second sequence when the relevant distances, under consideration of the scale factor, differ by more than a predetermined second measure.

Alternative plausibility supervisions can run as follows, according to embodiments of the invention:

before the association of the first sequence of the N markers with the second sequence of the K markers, scaling of one of the two coordinate systems with the scale factor, such that the distances between two coordinates in the first coordinate system are equal to the distance of the corresponding coordinates in the second coordinate system.

determination of a first vector in the possibly scaled first coordinate system based on the coordinates of two markers of the first sequence,

determination of a second vector in the possibly scaled first coordinate system based on the coordinates of two markers of the second sequence corresponding to the first vector,

determination of a translation and rotation that describes a transformation of the first vector of the possibly scaled first coordinate system into the second vector of the possibly scaled second coordinate system,

application of the transformation on the N marker points of the first sequence,

comparison of the transformed coordinates with the corresponding coordinates of corresponding markers of the second sequence, and

generation of an error message when at least one comparison is exceeded by more than a fourth measure.

However, it is also possible, as is provided according to a further embodiment xxx of the invention, to implement the following method steps before the association of the first sequence of the N markers with the second sequence of the K markers for a plausibility supervision:

a) determination of a first rotational direction and a first angle between a third vector and a fourth vector, whereby the third and fourth vectors are formed from coordinates of three successive markers of the first sequence in the first coordinate system, such that the third vector is based on the coordinates of the first two markers and the fourth vector is based on the coordinates of the last two markers,

b) determination of a second rotational direction and a second angle between a fifth vector and a sixth vector, whereby the fifth and sixth vectors are formed from coordinates of three successive markers of the second sequence in the second coordinate system, such that the fifth vector is based on the coordinates of the first two markers and the sixth vector is based on the coordinates of the last two markers,

c) comparison of the first rotational direction with the second rotational direction, and comparison of the first angle with the second angle,

d) generation of an error message when both rotational directions and/or when both angles differ by a predetermined fifth measure, and

e) application of the method steps a) through d) on vectors that are determined via the remaining markers of the first and the second sequence.

Based on the association of the first sequence of the N markers with the second sequence of the K markers, a registration matrix is calculated according to an embodiment of the invention.

The third object of the invention is achieved via a use of the inventive method for navigation of a medical instrument relative to the body of the subject, whereby the mapping of the medical instrument can be blended in the image of the patient.

The third object is also achieved via a use of the inventive method for superimposition of the first and second image.

The methods are particularly provided for use in medicine. The navigation of an instrument relative to the body of a patient represents a particularly advantageous field of use. A mapping of the instrument can be mixed into the image of the patient and shifted in the image, for example, by a doctor using a suitable operator device, for example a joystick, a trackball, or an actual navigation system.

Based on the unambiguous mathematical connection (determined with the aid of the inventive method) between the virtual situation shown in the image and the real situation prevailing on the patient, the doctor can navigate one or even multiple medical instruments relative to the patient, i.e., orient instruments relative to the patient corresponding to the situation shown in the image, or even simulate specific operative measures. For example, this could include the penetration of puncture needle into the body of a patient. Using the image information and the puncture needle mixed into the image, the doctor can, for example, find the suitable penetration location on the body surface of the patient and determine the corresponding location on the patient body via the established coordinate transformation. The image information can exist in the form of 2D or (preferably) 3D images of the patient, which can be determined and displayed with known imaging systems.

### DESCRIPTION OF THE DRAWINGS

Exemplary embodiments of the invention are exemplarily shown in the attached schematic drawings.

FIG. 1 is a pictorial illustration of markers arranged on a patient and markings mapped in an image, and

FIG. 2 is a pictorial illustration of further markers arranged on a patient and makings mapped in an image.

### DETAILED DESCRIPTION OF THE PREFERRED EMBODIMENTS

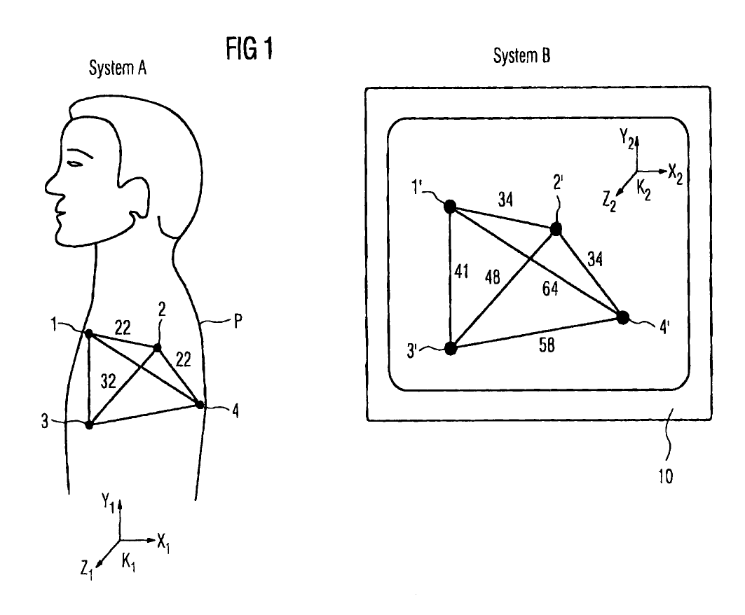

The system A comprises a patient P (indicated only schematically in FIG. 1) which is provided with N=4 markers. In the case of the present exemplary embodiment, the markers 1 through 4 are located on the body surface of the patient P. However, the markers 1 through 4 do not necessarily have to be located on the body surface of the patient P; rather, they can also be arranged in a suitable manner inside the body of the patient P. The markers 1 through 4 are preferably attached at locations that are supported by bones and optimally move little, for example, during the breathing of the patient P. These locations are, for example, the forehead, the pelvic bones, stationary ribs, or the sternum of the patient P.

The system B comprises a display device 10 on which a 3D volume image of the patient P can be displayed. Known imaging systems such as x-ray systems, MR systems, ultrasound systems, computer tomographs, etc. can be used to acquire the 3D volume image. The four markers 1 through 4 arranged on the patient P are fashioned such that, dependent on the imaging system used, they appear as contrast points, i.e., as K=4 mappings of the markers 1 through 4 in the 3D volume image of the patient P shown on the display device 10. Suitable markers are, for example, flat aluminum markers with holes. In the case of the present exemplary embodiment, for reasons of clarity, a representation of details has been omitted from the inside of the body of the patient of the 3D volume image shown on the display device 10. Rather, for explanation of the inventive method, only the markers 1′ through 4′ mapped in the 3D volume image are shown.

In order to be able to describe the mapping of the markers 1 through 4 of the system A to the mapped markers 1′ through 4′ of the system B and vice versa, the knowledge of which mapped markers 1′ through 4′ of the system B correspond to which markers 1 through 4 in the system A is necessary.

For this, the coordinates of the arbitrarily numbers markers 1 through 4 arranged on the patient P are inventively determined in the system A with regard to a first coordinate system K1 associated with the system A. In the case of the present exemplary embodiment, the coordinate system K1 is a Cartesian coordinate system, which is, however, not necessary. For the determination of the coordinates in the coordinate system K1, for example, the markers 1 through 4 arranged on the patient are individually tapped with, for example, a navigation system known from German Patent Document No. DE 199 51 502 A1.

Using the coordinates of the markers 1 through 4, calculations are performed to determine all distances between the markers 1 through 4. For each of the markers 1 through 4, the sum of the distances is subsequently calculated relative to the remaining markers 1 through 4. This means that, for the marker 1, a distance sum results from the distances between the marker 1 and 2, the marker 1 and 3 and the marker 1 and 4. The distance sum associated with the marker 2 is the sum of the distances between the marker 1 and 2, the marker 2 and 3 and the marker 2 and 4. For the marker 3, the corresponding distance sum is the sum from the distances between the markers 3 and 1, the markers 3 and 2, and the markers 3 and 4. For the marker 4, a distance sum results from the distances between the markers 4 and 1, the markers 4 and 2, and the markers 4 and 3.

In the case of the present exemplary embodiment, the distance sums are subsequently sorted in ascending order, meaning the sequence begins with the smallest distance sum and ends with the largest distance sum. Since each of the distance sums is associated with one of the markers 1 through 4, a first sequence (M₁,M₂,M₃,M₄) likewise results from this sequence for the individual markers 1 through 4 arranged on the patient P. In FIG. 1, the distances are moreover indicated between the markers 1 and 2, 3 and 4 and 4 and 2. The sequence can be summarized as follows:

(M₁,M₂,M₃,M₄)=(2,1,3,4)

The coordinates of the K markers 1′ through 4′ mapped in the image are subsequently determined. The mapped markers 1′ through 4′ can either be manually detected in arbitrary sequence, for example tapped with a cursor, or be automatically detected by way of a pattern recognition method. The sums of the distances of each marker 1′ through 4′ mapped in the image to each of the remaining mapped markers 1′ through 4′ is subsequently calculated. This means that a distance sum results for the mapped marker 1′ from the distances between the mapped marker 1′ and 2′, the mapped marker 1′ and 3′, and the mapped marker 1′ and 4′. The distance sum associated with the mapped marker 2′ is the sum of the distances between the mapped marker 1′ and 2′, the mapped marker 2′ and 3′, and the mapped marker 2′ and 4′. For the mapped marker 3′, the corresponding distance sum is the sum from the distances between the mapped markers 3′ and 1′, the mapped markers 3′ and 2′, and the mapped markers 3′ and 4′. For the mapped marker 4′, a distance sum results from the distances between the mapped markers 4′ and 1′, the mapped markers 4′ and 2′, and the mapped markers 4′ and 3′. In FIG. 1, the distances are also shown between the coordinates of the mapped markers 1′ through 4′ in the coordinate system K2.

In the case of the present exemplary embodiment, the distance sums are subsequently sorted in ascending order, meaning the sequence begins with the smallest distance sum and ends with the largest distance sum. Since each of the distance sums is associated with one of the mapped markers 1′ through 4′, a second sequence (M′₁,M′₂,M′₃,M′₄) likewise results from this sequence for the individual markers 1′ through 4′ mapped in the image. The sequence of the mapped markers 1′ through 4′ of the system B is summarized as follows in the case of the present exemplary embodiment:

(M′₁,M′₂,M′₃,M′₄)=(2′,1′,3′,4′)

In this manner, an unambiguous association of the markers 1 through 4 of the system A with the markers 1′ through 4′ mapped in the 3D volume image of the system B is obtained. The association is thereby based on the unambiguous determination of both sequences (M₁,M₂,M₃,M₄) and (M′₁,M′₂,M′₃,M′₄).

In order to test the association for plausibility, in the case of the present exemplary embodiment, the following method steps are executed:

First, a scale factor is formed from the quotients from the largest separation between two markers in the coordinate system K1 and the largest distance between two mapped markers in the coordinate system K2. Each individual distance sum that is associated with the markers 1′ through 4′ mapped in the image are subsequently multiplied with the scale factor and compared with the corresponding distance sum of the markers 1 through 4 arranged on the patient P. In the case of the present exemplary embodiment, the comparison is implemented in that the relevant distance sums are first subtracted from one another and absolute values of the individual results are acquired. It is subsequently tested as to whether each of the absolute values is smaller than a predetermined number. If this is the case, the association is plausible, and the transformation matrix that transforms the coordinates of the coordinate system K1 of the system A into the coordinates of the coordinate system K2 of the system B is calculated in a known manner.

If any of the absolute values is larger than the predetermined number, the association is not plausible, and an error message is generated that is mixed into the image.

A further plausibility check, in which the coordinate systems do not necessarily have to be scaled, can run as follows:

First, the rotational direction and the angle are determined between the vector that is formed by the coordinates of the markers 2 and 3 in the coordinate system K1 and the vector that is formed by the coordinates of the markers 3 and 4 in the coordinate system K1. The rotational direction and the angle is subsequently determined between the vector that is formed by the coordinates of the mapped markers 2′ and 3′ in the coordinate system K2 and the vector that is formed by the coordinates of the mapped markers 3′ and 4′. If both rotational directions are equal and both angles deviate by less than a predetermined measure, then the association is plausible. Otherwise, an error message is again generated.

These steps are also applied to the remaining coordinates associated with the markers or the mapped markers.

In the case of the exemplary embodiment explained using FIG. 1, N=4 markers are arranged on the patient and K=4 markers are mapped in the image. However, it is possible that more or fewer markers are arranged on the patient P than are mapped in the image since, for example, a marker arranged on the patient is not visible in the image or has come loose in the registration.

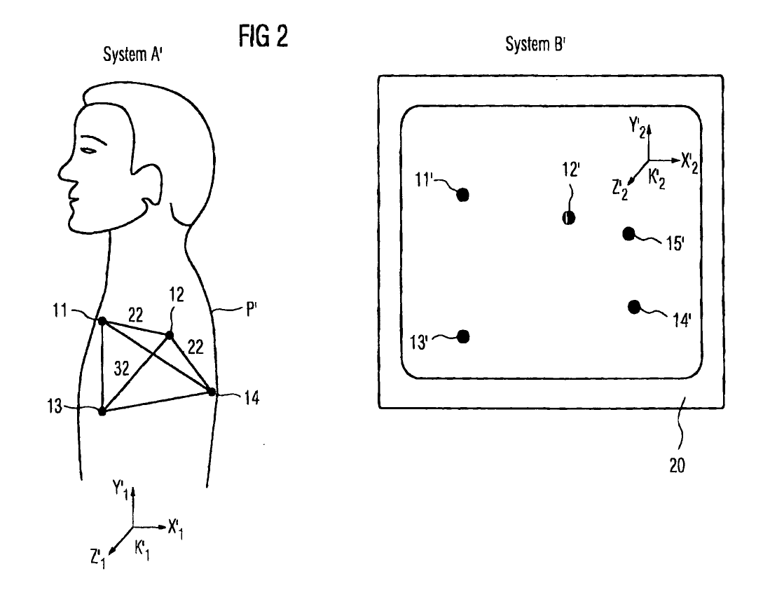

A case in which N≠K is schematically shown in FIG. 2. The system A′ comprises a patient P′ (shown only schematically in FIG. 2) which is provided with N′=4 markers 11 through 14. In the case of the present exemplary embodiment, the markers 11 through 14 are located on the body surface of the patient P′.

The system B′ comprises a display device 20 on which a 3D volume image of the patient P′ can be displayed. Known imaging systems such as x-ray systems, MR systems, ultrasound systems, computer tomographs, etc. can be used to acquire the 3D volume image. In the acquisition of the 3D volume data set, in addition to the markers 11 through 14 arranged on the patient P, a fifth marker is applied, such that K′=5 mapped markers 11′ through 15′ appear in the 3D volume image of the patient P′ shown on the display device 20.

In order to be able to describe the mapping of the markers 11 through 14 of the system A′ to the mapped markers 11′ through 15′ of the system B′ and vice versa, the knowledge of which markers 11′ through 15′ of the system B′ mapped in the 3D volume image correspond to which markers 11 through 14 in the system A′ is necessary.

For this, the coordinates of the arbitrarily numbers markers 11 through 14 arranged on the patient P′ are inventively determined in the system A′ with regard to a first coordinate system K1′ registering the system A′ as explained above. Using the coordinates of the markers 11 through 14, all distances related to the distance of markers 11 through 14 from one another are calculated. For each of the markers 11 through 14, the sum of the distances is subsequently calculated relative to the remaining markers 11 through 14, as explained above.

In the case of the present exemplary embodiment, the distance sums are subsequently sorted in ascending order, meaning the sequence begins with the smallest distance sum and ends with the largest distance sum. Since each of the distance sums is associated with one of the markers 1 through 4, a sequence (M₁₁,M₁₂,M₁₃,M₁₄) likewise results from this sequence for the individual markers 11 through 14 arranged on the patient P′. The sequence of the markers 11 through 14 of the system A′ is, in the case of the following exemplary embodiment, summarized as follows:

(M₁₁,M₁₂,M₁₃,M₁₄)=(12,11,13,14)

The coordinates of the K′ markers 11′ through 15′ mapped in the image are subsequently determined as already explained. A sequence of the mapped markers 11′ through 15′ of the system B′ that results is, in the case of the following exemplary embodiment, summarized as follows:

(M′₁₁,M′₁₂,M′₁₃,M′₁₄,M′₁₅)=(12′,15′,14′,11′,13′)

Since N′ is now ≠K′, it must first be determined which of the markers 11 through 14 arranged on the patient P′ are mapped in the image. For this purpose, partial sequences are formed from the sequence of the system B′ associated with the mapped markers, in that (in the case of the present exemplary embodiment) respectively one mapped marker is removed; the number of mapped markers of each partial sequence is thus equal to the number of the markers arranged on the surface of the patient P′. The distances are subsequently determined for each partial sequence of the distances of the coordinates associated with the individual markers, distance sums are formed from each marker to the remaining markers of the corresponding partial sequence, and the partial sequences are sorted according to ascending values. In the case of the present exemplary embodiment, the following partial sequences thus result:

(M′₁₂,M′₁₃,M′₁₄,M′₁₅)=(15′,14′,11′,13′),

(M′₁₁,M′₁₃,M′₁₄,M′₁₅)=(12′,11′,13′,14′),

(M′₁₁,M′₁₂,M′₁₄,M′₁₅)=(12′,11′,15′,13′),

(M′₁₁,M′₁₂,M′₁₃,M′₁₅)=(15′,12′,14′,13′),

(M′₁₁,M′₁₂,M′₁₃,M′₁₄)=(12′,15′,14′,11′).

For each partial sequence, a correlation coefficient rₙ is subsequently determined, in that, respectively, the distance sums of a partial sequence correlates with the corresponding distance sums of the sequence of system A′ which is associated with the markers 11-14 arranged on the patient P′:

r₁: (15′,14′,11′,13′) correlates with (12, 11, 13, 14),

r₂: (12′,11′,13′,14′) correlates with (12, 11, 13, 14),

r₃: (12′,11′,15′,13′) correlates with (12, 11, 13, 14),

r₄: (15′,12′,14′,13′) correlates with (12, 11, 13, 14),

r₅: (12′,15′,14′,11′) correlates with (12, 11, 13, 14).

That partial amount that is associated with the largest correlation coefficient rₙ comprises those markers mapped in the 3D volume image that are also arranged on the patient P′. In the case of the present exemplary embodiment, this is the partial sequence with r₂, thus (12′,11′,13′,14′).

In this manner, an unambiguous association of the markers 11 through 14 of the system A′ with the markers mapped in the 3D volume image is obtained. In the case of the present exemplary embodiment, the markers 11 through 14 arranged on the patient P′ correspond with the markers 11′, 12′, 13′ and 14′ mapped in the 3D volume image. The association is thereby based on the unambiguous determination of both sequences (M₁₁,M₁₂,M_{13,}M₁₄) and (M′₁₁,M′₁₂,M′₁₃,M′₁₄). The transformation matrix that transforms the coordinates of the coordinate system K1′ of the system A′ into the coordinates of the coordinate system K2′ of the system B′ is therefore calculated in a known manner.

In the case of the present exemplary embodiment, associations of markers applied on a patient with markers mapped in an image have been described. The inventive method can also be applied to the association of markers mapped in two images.

The application of the inventive method is provided in medicine, particularly for navigation of a medical instrument relative to the body of a patient. A doctor can thereby change the 3D volume image of the patient P (which is shown by an image computer of the imaging system on the display device 10 or 20) in terms of its position by way of known operating devices, for example, a joystick, a trackball, or a real navigation system and, for example, mix mappings of medical instruments which an examiner would like to use on the patient. The representation of the patient in the form of a 3D volume image offers the examiner the possibility to, for example, find the optimal start point of an instrument for an operative procedure, for example the optimal start location of a puncture needle relative to the body surface of the patient, and to correspondingly orient the organ to be punctured. With the aid of the acquired mapping instruction, the examiner obtains the coordinates and the orientation of the instrument as it must be applied for the optimal procedure on patient P in system A.

The inventive method can particularly also be used for superimposition of the two images.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The present invention may be described in terms of functional block components and various processing steps. Such functional blocks may be realized by any number of hardware and/or software components configured to perform the specified functions. For example, the present invention may employ various integrated circuit components, e.g., memory elements, processing elements, logic elements, look-up tables, and the like, which may carry out a variety of functions under the control of one or more microprocessors or other control devices. Similarly, where the elements of the present invention are implemented using software programming or software elements the invention may be implemented with any programming or scripting language such as C, C++, Java, assembler, or the like, with the various algorithms being implemented with any combination of data structures, objects, processes, routines or other programming elements. Furthermore, the present invention could employ any number of conventional techniques for electronics configuration, signal processing and/or control, data processing and the like.

The particular implementations shown and described herein are illustrative examples of the invention and are not intended to otherwise limit the scope of the invention in any way. For the sake of brevity, conventional electronics, control systems, software development and other functional aspects of the systems (and components of the individual operating components of the systems) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

### REFERENCE LIST

1-4, 1′-4′, 11-14, 11′-15′ markers

| | |
|---|---|
| 10, 20 | display device |
| A, B, A′, B′ | system |
| K1, K1′, K2, K2′ | coordinate system |
| P, P′ | patient |

What is claimed is:

1. A method for associating a set O of N markers arranged on a subject and a set Q of K markers mapped in an image of the subject relative to one another, comprising:
determining coordinates of the N markers arranged on the subject in a first coordinate system;
determining a first set of distance sums in which each respective distance sum of each marker arranged on the subject is added to each of the remaining N−1 markers arranged on the subject;
sorting the first set of distance sums in an order that is either ascending or descending;
determining a first sequence of the N markers arranged on the subject based on the ascending or descending order of the distance sums of the first set of distance sums;
determining coordinates of the K markers mapped in the image in a second coordinate system;
determining a second set of distance sums in which each respective distance sum of each marker mapped in the image is added to each of the remaining K−1 markers mapped in the image;
sorting the second set of distance sums in an order that is either ascending or descending;
determining a second sequence of the K markers mapped in the image based on the ascending or descending order of the distance sums of the second set of distance sums;
if N=K, then associating the first sequence of the N markers with the second sequence of the K mapped markers; and
utilizing this association in a medical or technical application.

2. The method according to claim 1, wherein the orders are ascending.

3. The method according to claim 1, wherein the orders are descending.

4. A method for association of a set O of N markers mapped in a first image and a set Q of K markers mapped in a second image relative to one another, whereby both images are mappings of a subject and the markers are arranged on the subject during exposures and are mapped in both images, comprising:
determining coordinates of the N markers mapped in the first image in a first coordinate system,
determining a first set of distance sums in which each respective distance sum of each marker mapped in the first image is added to each of the remaining N−1 markers mapped in the first image, and
sorting the first set of distance sums in an order that is either ascending or descending;
determining a first sequence of the N markers mapped in the first image based on the ascending or descending order of the distance sums of the first set of distance sums.
determining coordinates of the K markers mapped in the second image in a second coordinate system;
determining a second set of distance sums in which each respective distance sum of each marker mapped in the second image is added to each of the remaining K−1 markers mapped in the second image, and
sorting the second set of distance sums in an order that is either ascending or descending;
determining a second sequence of the K markers mapped in the second image based on the ascending or descending order of the distance sums of the second set of distance sums;
if N=K, then associating the first sequence of the N mapped markers with the second sequence of the K mapped markers; and
utilizing this association in a medical or technical application.

5. The method according to claim 4, wherein the orders are ascending.

6. The method according to claim 4, wherein the orders are descending.

7. The method according to claim 1, wherein when N<K, the method further comprises:
forming partial sequences from the second sequence, the number of mapped markers of each partial sequence being equal to N;
determining distances between coordinates for each two adjacently mapped markers of the partial sequences;
determining correlation coefficients for each of the partial sequences with the first sequence, in that respectively distances between two mapped markers of a partial sequence are correlated with corresponding distances between two markers or between two mapped markers of the first sequence, and
associating the first sequence with that partial sequence having the largest correlation coefficients.

8. The method according to claim 1, wherein when N>K, the method further comprises:
forming partial sequences from the second sequence, the number of the markers or mapped markers of each partial sequence being equal to K;
determining distances between coordinates for each two adjacently mapped markers or between two mapped markings of the partial sequences;
determining correlation coefficients for each of the partial sequences, in that respectively the distances between two markers or mapped markers of a partial sequence are correlated with the corresponding distances between two mapped markers of the second sequence; and
associating the second sequence with that partial sequence with the largest correlation coefficients.

9. The method according to claim 1, wherein when N<K, the method further comprises:
forming partial sequences from the second sequence, the number of mapped markers of each partial sequence being equal to N;
determining distances between coordinates for each two adjacently mapped markers of the partial sequences;
determining correlation coefficients for each of the partial sequences, in that respectively the sorted distance sums of mapped markers of a partial sequence are correlated with the sorted distance sums of the markers or of the mapped markers of the first sequence; and
associating that partial sequence with the largest correlation coefficients with the first sequence.

10. The method according to claim 1, wherein when N>K, the method further comprises:
forming partial sequences from the first sequence, the number of markers or mapped markers of each partial sequence being equal to K;
determining distances between coordinates for each two adjacently mapped markers of the partial sequences;
determining a correlation coefficient for each of the partial sequences, in that respectively the sorted distance sums of the markers or mapped markers of a partial sequence are correlated with the sorted distance sums of mapped markers of the first sequence; and
associating that partial sequence with the largest correlation coefficients with the second sequence.

11. The method according to claim 1, wherein for points having a same distance sum K, the method further comprises:
determining a first point with a distance sum occurring only once;
sorting ambiguous points in monotonically increasing distance from the first point; and
when such a distance value likewise occurs multiple times, sorting of the ambiguous points such that the angles of the straight lines from the first point to the ambiguous points changes monotonically in the final sorting in a specific mathematical system.

12. The method according to claim 1, the method further comprising, after determining the second set of distance sums:
determining a scale factor between both coordinate systems which expresses a magnitude ratio between the distances of two coordinates in one coordinate system and in the other coordinate system.

13. The method according to claim 12, wherein when N=K and under consideration of the scale factor, the method further comprises:
comparing corresponding distance sums of both sets of distance sums; and
generating an error message when at least one pair of corresponding distance sums differs by more than a predetermined first measure.

14. The method according to claim 12, wherein when N<K, the method further comprises:
starting from the first sequence and taking into account the scale factor, successively comparing the distances of two successive markers of the first sequence with the distances of subsequent markers of the second sequence; and
removing current subsequent markers from the second sequence when the relevant distances, under consideration of the scale factor, differ by more than a predetermined second measure.

15. The method according to claim 12, wherein when N>K, the method further comprises:
starting from the second sequence and taking into account the scale factor, successive comparing the distances of two successive markers of the second sequence with the distances of subsequent markers of the first sequence; and
removing current subsequent markers from the first sequence when the relevant distances, under consideration of the scale factor, differ by more than a predetermined third measure.

16. The method according to claim 12, further comprising, before the association of the first sequence of the N markers with the second sequence of the K markers:
scaling of one of the two coordinate systems with the scaling factor, such a distance between two coordinates in the first coordinate system are equal to a distance of the corresponding coordinates in the second coordinate system;
determining a first vector in the scaled or unscaled first coordinate system based on coordinates of two markers of the first sequence;
determining a second vector in the scaled or unscaled first coordinate system based on coordinates of two markers of the second sequence corresponding to the first vector;
determining a translation and rotation that describes a transformation of the first vector of the scaled or unscaled first coordinate system into the second vector of the scaled or unscaled second coordinate system;
applying the transformation to the coordinates of all N markers of the first sequence;
comparing the transformed coordinates with corresponding coordinates of corresponding markers of the second sequence; and
generating an error message when at least one comparison is exceeded by more than a fourth measure.

17. The method according to claim 1, further comprising, before associating the first sequence of the N markers with the second sequence of the K markers:
a) determining a first rotational direction and a first angle between a third vector and a fourth vector, the third and fourth vectors being formed from coordinates of three successive markers of the first sequence in the first coordinate system, such that the third vector is based on the coordinates of the first two of these markers and the fourth vector is based on the coordinates of the last two of these markers,
b) determining a second rotational direction and a second angle between a fifth vector and a sixth vector, the fifth and sixth vectors being formed from coordinates of three successive markers of the second sequence in the second coordinate system, such that the fifth vector is based on the coordinates of the first two of these markers and the sixth vector is based on the coordinates of the last two of these markers,
c) comparing the first rotational direction with the second rotational direction, and comparing the first angle with the second angle,
d) generating an error message when at least one of both rotational directions and both angles differ by a predetermined fifth measure; and
e) applying steps a) through d) to vectors that are determined via remaining markers of the first and the second sequence.

18. The method according to claim 1, further comprising forming a registration matrix from the association of the first sequence of the N markers with the second sequence of the K markers.

19. The method according to claim 1, wherein the medical or technical application comprises navigating a medical instrument relative to a body of the subject, a mapping of the medical instrument being mixed into an image of the patient.

20. The method according to claim 4, wherein the medical or technical application comprises superimposing a first and a second image.
